(19) United States
(12) Patent Application Publication
(10) Pub. No.: US 2018/0064347 A1
(43) Pub. Date: Mar. 8, 2018

(54) HANDHELD DEVICE AND MULTIMODAL CONTRAST AGENT FOR EARLY DETECTION OF HUMAN DISEASE

(71) Applicant: The Penn State Research Foundation, University Park, PA (US)

(72) Inventors: JAMES H. ADAIR, STATE COLLEGE, PA (US); SEAN D. KNECHT, STATE COLLEGE, PA (US); J. ERIC BOYER, UNIVERSITY PARK, PA (US); RICHARD L. TUTWILER, PORT MATILDA, PA (US); CONNOR CARR, UNIVERSITY PARK, PA (US); XIAOMENG TANG, UNIVERSITY PARK, PA (US); BERNADETTE M. ADAIR, UNVERSITY PARK, PA (US); THOMAS NEUBERGER, STATE COLLEGE, PA (US); WELLEY S. LOC, UNIVERSITY PARK, PA (US); ZACHARY R. WILCZYNSKI, UNIVERSITY PARK, PA (US); CHRISTOPHER MCGOVERN, HARRISBURG, PA (US); GAIL L. MATTERS, HUMMELSTOWN, PA (US); KEITH CHENG, HERSHEY, PA (US); MARK KESTER, HARRISBURG, PA (US); LAWRENCE SINOWAY, HERSHEY, PA (US)

(21) Appl. No.: 15/699,215

(22) Filed: Sep. 8, 2017

Related U.S. Application Data
(60) Provisional application No. 62/384,849, filed on Sep. 8, 2016.

(51) Int. Cl. A61B 5/00; A61K 49/22

(52) U.S. Cl. CPC A61B 5/0095; A61B 2503/40; A61K 49/221; A61K 49/222

(57) ABSTRACT

Systems comprising a combination of the handheld imaging system with a nanoparticle multimodal contrast agent are disclosed. The imaging system exploits the advantages of both near-infrared emission and the photoacoustic effect by employing calcium phosphosilicate nanocolloid that encapsulates NIR and CT/MRI contrast agents for enhanced deep tissue imaging as well as a portable NIR/PA system using a tunable pulsed laser, CCD imaging technology and acoustic transducer arrays. Methods for using the system, for example in rapid diagnosis of trauma such as that inflicted on a battlefield, are provided.

### CROSS-REFERENCE TO RELATED APPLICATIONS

This application claims priority and is related to U.S. Provisional Application Ser. No. 62/384,849 filed on Sep. 8, 2016 and entitled “HANDHELD DEVICE AND MULTIMODAL CONTRAST AGENT FOR EARLY DETECTION OF HUMAN DISEASE.” The entire contents of this patent application are hereby expressly incorporated herein by reference including, without limitation, the specification, claims, and abstract, as well as any figures, tables, or drawings thereof.

### GRANT INFORMATION

This invention was made with government support under Grant No. CA167535, awarded by the National Institutes of Health. The Government has certain rights in the invention.

### FIELD OF THE DISCLOSURE

The disclosure relates to systems comprising a combination of a handheld imaging system with a nanoparticle multimodal contrast agent. The imaging system exploits the advantages of both near-infrared emission and the photoacoustic effect by employing calcium phosphosilicate nanocolloid that encapsulates NIR and CT/MRI contrast agents for enhanced deep tissue imaging as well as a portable NIR/PA system using a tunable pulsed laser, CCD imaging technology and acoustic transducer arrays. Methods for using the system, for example in rapid diagnosis of trauma such as that inflicted on a battlefield, are provided.

### BACKGROUND OF THE DISCLOSURE

Rapid diagnosis and treatment of trauma by medical professional is the key element for survival of wounded patients. This is particularly true for battle field trauma. Battlefield medics for trauma treatment are exceptionally well-trained military healthcare personnel, serving all branches of U.S. armed forces. The quality of care for battlefield trauma is intimately connected with the deployment of sophisticated, but robust diagnostic instruments for rapid diagnosis and triaging prior to transporting wounded personnel to the combat surgical care center. For example, in Operation New Dawn (OND), Operation Iraqi Freedom (OIF) and Operation Enduring Freedom (OEF), acoustic ultrasound devices that are at least transportable, if not wholly portable, have been used for rapid battle field diagnosis of internal trauma. While the ultrasound systems have permitted rapid diagnosis, issues with the portability (the system is about the size of a larger suitcase), detection limits, operator dependence, obscuration by wound dressings, and lateral resolution remain issues that require improvement.

Data recently compiled by those in the field demonstrates the need for more sophisticated and sensitive detection of both head trauma and internal bleeding associated with the torso, axilla, and extremities. During the course of OND, OEF, and OIF (as of Feb. 5, 2013), there have been 6,604 service member deaths and 50,450 service members wounded in action. There have been multiple studies during the past twenty years evaluating the cause of death from combat injuries and establishing whether service members could have potentially survived. For example, the study conducted by Eastridge et al., found that out of 558 service personnel who died due to wounds that about 51% of the service member deaths were potentially survivable. The analyses of the post-mortem autopsies indicated that 41% of the total 558 deaths due to wounds were caused by hemorrhage-major trauma associated with, from high to low incidence, torso, extremity, and junctional regions.

The epidemiology data collected in the various studies during the last two decades underscore the need for diagnostic devices with real time capability and greater portability on the modern battlefield that can be used by the medical personnel trained for such a chaotic environment. Furthermore, more nimble diagnoses must be accompanied by more rapid treatment. The DARPA Stasis program has been addressing the latter need, with the prime example the ‘stasis foam’ developed by Arsenal Medical that is designed to stabilize internal hemorrhaging long enough for transport to the combat field hospital. The stasis foam approach has created great excitement in both the scientific community as well as the popular press and has recently received DARPA funding to move through FDA clinical trials.

Photonic techniques have the potential to address the issues associated with rapid diagnoses. In fact, a hand-held, near infra-red (NIR) device was developed to evaluate subdural traumatic head injuries and approved by the U.S. Food and Drug Administration in 2011. The use of near-infrared (NIR) radiation in medical imaging is a well-known method including the use of fluorescent particles and dyes, such as indocyanine green (ICG), to identify biological structures or behavior. These dyes or particles can be injected into the body which is then illuminated with NIR light near the excitation frequency of the particle/dye, a fluorescence emission of light at a longer wavelength, due to the Stokes shift, is produced which can then be imaged with a detector such as a charge-coupled device (CCD). The strength of the fluorescence signal is proportional to the input irradiance and the number of fluorescing particles. FDA-approved ICG, also known as Cardio Green, has an excitation peak around 785 nm in dilute aqueous solution with the fluorescence spectrum peak above 830 nm. The values can vary based on the ICG concentration and the local environment. These are convenient NIR wavelengths for biological use due to their spectral peaks existing in the so-called NIR therapeutic window, a wavelength region (≈700 nm-1 μm) which exhibits deeper penetration into soft tissue. The relative transparency of NIR photons through soft tissue has engendered great activity during the last 20 years to understand and develop photon-based imaging modalities. Applicant's research according to the present disclosure indicate an effective fluorescence/absorbance/transmission of NIR at a wavelength of 785 nm and emitted photons greater than 830 nm can be detected in real time, vis-à-vis, time lapse imaging, from 1 cm to about 3 cm deep in soft tissue.

Nevertheless, the NIR radiation from both the external source and the fluorescence of the dye are subject to attenuation, primarily due to scattering events. Thus, imaging in deeper regions of the body is limited by the power of the external source, the number of fluorescing particles, the quantum efficiency of the fluorophores (number of fluorescence photons per incident photon) and the sensitivity of the detector. However, for the detection of internal hemorrhaging without extended time to collect a significant number of photons, information from deeper within the human body than that afforded by NIR techniques is required to establish the anatomical volume associated with major trauma. Furthermore, the imaging should be compatible with potential deployment of treatment schemes such as the stasis foam to work in concert with this and other innovative approaches to not only detect the region of trauma, but also to treat the major trauma and internal hemorrhaging. Additionally, the imaging approach should be compatible with the X-ray computer tomography scans (CT) used to rapidly diagnose wounds in the combat field hospital and the slower, but even more highly resolved, three-dimensional imaging provided by magnetic resonance imaging (MRI) used in the wound treatment centers for military service members.

The use of multi-imaging modality nanoparticles according to the present disclosure permits the engineering of improved quantum efficiency fluorophores into the nanoparticle design optimizing the signal, along with CT and MRI contrast agents. In a battlefield situation, the use of the NIR imaging mode would be of greatest use in quickly identifying the general location of internal bleeding by imaging “hot-spots” from the fluorescence emission of the nanoparticles. Higher resolution of internal bleeding sites, as well as particularly deep injuries, will require an additional imaging modality: photoacoustic imaging.

The rapid diagnosis of internal hemorrhage due to blast, crush or blunt trauma is still severely limited by the sensitivity of conventional handheld ultrasound or near infrared imaging devices. New modalities that offer increased sensitivity are urgently needed. The innovation of the present disclosure is the use of photoacoustic imaging, which combines the high contrast of light absorption with the improved depth imaging of ultrasound. This dual-imaging modality of the present disclosure significantly improves real-time diagnosis of internal injuries on the battlefield.

Accordingly, it is an objective of the claimed disclosure to develop a handheld device employing a multimodal contrast agent for early and rapid detection of disease and trauma.

A still further object is to develop a nanoparticle bioimaging contrast agent for multimodal biological imaging.

A further object of the disclosure is to improve depth imaging of tissue via ultrasound combined with high contrast light absorption.

A further object of the disclosure is to improve real-time diagnosis of internal injuries.

Other objects, advantages and features of the present disclosure will become apparent from the following specification taken in conjunction with the accompanying figures.

### SUMMARY OF THE DISCLOSURE

In an aspect of the disclosure, Applicant has developed a nanocolloid that encapsulates NIR and CT/MRI contrast agents for enhanced deep tissue imaging as well as a portable NIR/PA system using a tunable pulsed laser, CCD imaging technology and acoustic transducer arrays. The present disclosure provides numerous advantages over existing imaging technology such as ultrasound and CT, including but not limited to; rapidity of use and diagnosis; affordability; and availability.

The disclosure consists of three related embodiments: a handheld photoacoustic, portable imaging system; a nanoparticle bioimaging contrast agent for multimodal biological imaging; and the combination of the handheld imaging system with the nanoparticle multimodal contrast agent. The portability of the photoacoustic imaging is innovative and novel relative to the current state of development of imaging modality.

In one embodiment, the disclosure comprises a handheld photoacoustic portable imaging system. This device relies upon a dual modality, nontoxic, biocompatible nanocolloid that enhances photoacoustic tomographic imaging. In a preferred embodiment, the handheld photoacoustic portable imaging system comprises a tunable pulsed laser, a detector, and acoustic transducer arrays. In a more preferred embodiment, the handheld photoacoustic portable imaging system comprises CCD imaging technology. In another preferred embodiment, the handheld photoacoustic portable imaging system comprises a high-energy Nd:YAG pump laser system or a supercontinuum source. In a most preferred embodiment the handheld photoacoustic portable imaging system can be transported by a single individual.

In another embodiment, the disclosure comprises a nanoparticle bioimaging contrast agent for multimodal biological imaging. The agent is a dual modality, nontoxic, biocompatible nanocolloid that enhances photoacoustic tomographic imaging. In a preferred embodiment, the nanoparticle bioimaging contrast agent comprises pegylated calcium phosphosilicate nanoparticles. In a more preferred embodiment, the nanoparticle bioimaging contrast agent further comprises indocyanine green. In a more preferred embodiment, the nanoparticle bioimaging contrast agent further comprises nanoscale magnetite. In another embodiment, the nanoparticle bioimaging contrast agent is incorporated into a food additive or tablet, allowing the agent to be present in an individual when imaging in necessary without requiring additional administration.

In another embodiment, the disclosure comprises a method for rapidly detecting trauma in a person or animal, comprising administering to said individual or animal a nanoparticle bioimaging contrast agent for multimodal biological imaging; and imaging the distribution of said nanoparticle bioimaging contrast agent within said individual or animal using a handheld photoacoustic, portable imaging system. In a preferred embodiment, the nanoparticle bioimaging contrast agent comprises pegylated calcium phosphosilicate nanoparticles, indocyanine green and nanoscale magnetite. In another preferred embodiment, the administration comprises providing said individual or animal with a food additive or tablet comprising said nanoparticle bioimaging contrast agent. In another preferred embodiment, the handheld photoacoustic, portable imaging system comprises a tunable pulsed laser, a detector, and acoustic transducer arrays. In a more preferred embodiment, the handheld photoacoustic portable imaging system comprises CCD imaging technology. In another preferred embodiment, the handheld photoacoustic portable imaging system comprises a high-energy Nd:YAG pump laser system or a supercontinuum source.

In another embodiment, the disclosure comprises a system for rapid diagnosis of trauma comprising a handheld photoacoustic, portable imaging system and a nanoparticle multimodal contrast agent. In a preferred embodiment, the handheld photoacoustic, portable imaging system comprises a tunable pulsed laser, a detector, and acoustic transducer arrays, wherein the detector comprises CCD imaging technology, and the tunable laser comprises a high-energy Nd:YAG pump laser system or a supercontinuum source.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### DESCRIPTION OF THE FIGURES

FIG. 1 shows a diagrammatic representation of the presently claimed disclosure for detection of deep, soft tissue trauma and internal hemorrhaging based on Penn State's novel NIR-photoacoustic imaging and multimodal nanoparticle contrast agent.

FIG. 2 shows optical properties of NIR-CPSNPs. The top panel shows a photograph, and the bottom panel shows a graphical representation of NIR transmission through human tissue as a function of wavelength and the therapeutic ‘window’ associated with human tissue components is shown in.

FIG. 3 shows NIR transmission through porcine muscle tissue.

FIG. 4 shows the experimental configuration to determine the greater than 830 nm fluorescence from the NIR-CPSNPs. Bottom left shows NIR-CPSNP fluorescence in silicone tube embedded within ˜1 cm of porcine tissue and bottom right shows evidence for a pooling of NIR-CPSNP suspension under the porcine tissue.

FIG. 5 shows NIR detection and imaging of human breast cancer xenografts in a murine model for up to 96 hours after systemic tail vein injection of the PEG-NIR-CPSNP suspended in phosphated buffered saline.

FIG. 6 shows the design of multimodal, ensemble nanoparticulate for NIR-photoacoustic contrast, CT Scan, and MRI. (A) shows indo-cyanine green encapsulated in CPSNPs. (B) shows magnetite nanoparticles for CT Scan-MRI contrast. (C) shows ensemble nanocomposite, multimodal imaging nanoparticulates.

FIG. 7 shows a comparison of poorly dispersed magnetite nanoparticles to the PEG saturated surface that are well dispersed. (C)-(D): Particle size distributions confirm the poorly dispersed magnetite nanoparticles and well dispersed as do zeta potentials. (E)-(G): Standard spin echo MRI images of poorly dispersed (E) and well dispersed nanoparticles (F) in phantom acquired on a 14.1 Tesla MRI system. Top left image in both (E) and (F) is a control followed by five samples with increasing magnetite content (0.01, 0.025, 0.05, 0.08, and 0.1 mg/ml). The homogeneity is significantly greater for the well dispersed nanoparticles than the poorly dispersed. (G) shows T2 changes and relaxivity for the well dispersed particles at different concentrations.

FIG. 8 shows multispectral optoacoustic (MSOT) imaging of PEG-ICG-calcium phosphosilicate nanoparticles on the iThera Medical MSOT Small Animal Imaging System. (A) Phantom analysis—Blind unmixing. Software determines principal component of dataset and shows the near infra-red spectrum (Left) and corresponding image (Right); (B) Dilutions of ICG-particles placed in a phantom tissue sample; (Left) photoacoustic signals recorded from 680-900 nm, and (Right) multispectral unmixing by linear regression used to determine the lower limit of quantification: LLOQ<100 nM ICG.

FIG. 9 shows a system block diagram of the NIR-photoacoustic system based on a supercontinuum/tunable laser system combined with acoustic tomography.

FIG. 10 shows a laser system block diagram designed for the supercontinuum laser designed to receive timing commands from the operating system in FIG. 9.

FIG. 11 shows the signal processing chain of an exemplary embodiment of the disclosure.

FIG. 12 shows hybrid Portable Probe Design: (a) photoacoustic tomography (PAT) System, (b) NIR System.

FIG. 13 shows the integrated system architecture for hybrid system. Hand held imaging probe interfaced with miniaturized ultrasonic chip and transducer array.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION OF THE DISCLOSURE

The present disclosure relates to a nanocolloid that encapsulates NIR and CT/MIR contrast agent for enhanced deep tissue imaging and methods of systems of employing the same. The present disclosure further relates to a system comprising a combination of a handheld imaging system with a nanoparticle multimodal contrast agent and methods of employing the same. The compositions, systems, and methods have many advantages over existing imaging techniques. For example, the composition, systems and methods according to the disclosure provide rapid, deep tissue detection of blood pooling and internal trauma; portability of a laser excitation source; compatibility with other traditional treatments; and enhanced contrast for other imaging modalities.

The embodiments of this disclosure are not limited to particular compositions, methods, and/or systems which can vary and are understood by skilled artisans. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

### Definitions

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below. The term “about,” as used herein, refers to variation in the numerical quantity that can occur, for example, through typical measuring techniques and equipment, with respect to any quantifiable variable, including, but not limited to, mass, volume, time, distance, wave length, frequency, voltage, current, and electromagnetic field. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The term “microscale” and the related prefix “micro-” as used herein is intended to refer to items that have at least one dimension that is one or more micrometers and less than one millimeter.

The term “nanoscale” and the related prefix “nano-” as used herein is intended to refer to measurements that are less than one micrometer.

The term “nanoparticle” includes, for example, “nanospheres,” “nanorods,” “nanocups,” “nanowires,” “nanoclusters,” “nanofibers,” “nanolayers,” “nanotubes,” “nanocrystals,” “nanobeads,” “nanobelts,” and “nanodisks.”

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

As used herein, the phrases “medical instrument,” “dental instrument,” “medical device,” “dental device,” “medical equipment,” or “dental equipment” refer to instruments, devices, tools, appliances, apparatus, and equipment used in medicine or dentistry. Such instruments, devices, and equipment can be cold sterilized, soaked or washed and then heat sterilized, or otherwise benefit from cleaning in a composition of the present disclosure. These various instruments, devices and equipment include, but are not limited to: diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, scopes (e.g., endoscopes, stethoscopes, and arthoscopes) and related equipment, and the like, or combinations thereof.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

### Nanoparticle Compositions

Nanoparticulate contrast agents fall broadly into the following categories: compound semi-conductor known generally as Quantum Dots™; organic-based systems using nanoliposomes, cross-linked organics or some combination; and inorganic materials such as gold nanoshells and the calcium phosphosilicate nanoparticles described in U.S. Pat. No. 8,071,132, which is incorporated herein in its entirety. Nanoparticulate contrast agents generally are superior to molecular imaging agents. In summary, Quantum Dots™ are composed of toxic, heavy metal compound semi-conductors that have yet to be adapted or suitably modified for human use. Furthermore, it has been demonstrated that Quantum Dots™ are toxic and cause autophagy (a type of cell death) in porcine nephron endothelial cell culture and the autophagy is, unexpectedly, a product of the nanoparticle, not the heavy metal core material. Organic-based nanoparticulates, while generally biocompatible, begin to degrade immediately upon introduction into the circulatory system and the leaky nature and short times for circulation before complete degradation limits their use.

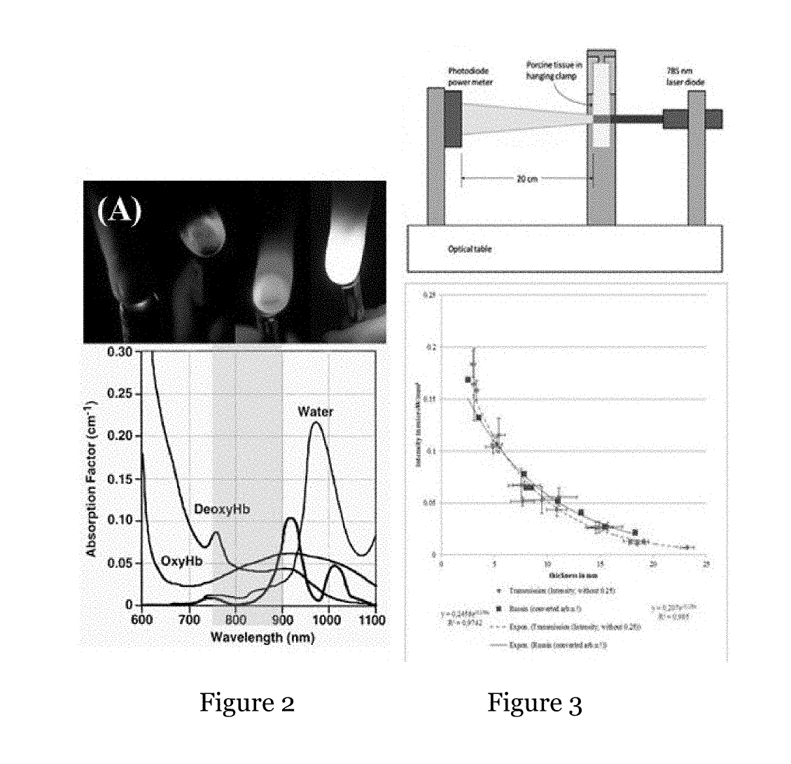

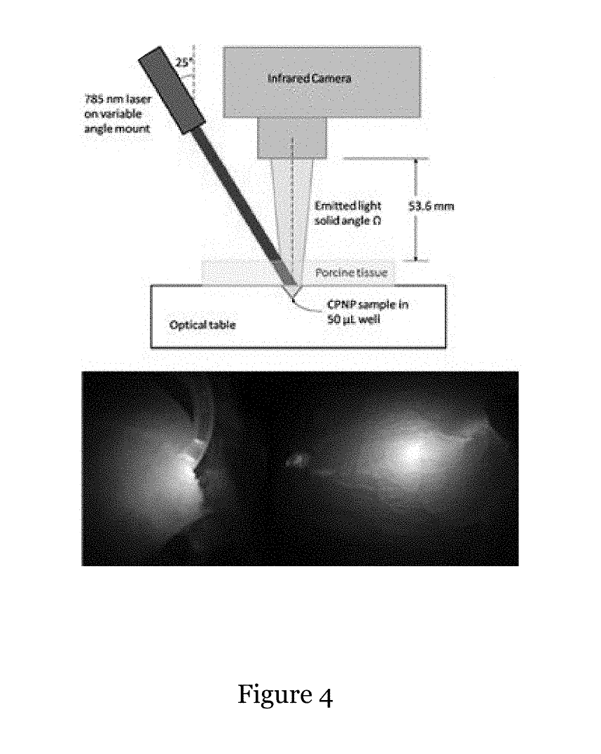

In contrast to other types of nanoparticles for bioimaging, the nanoparticles according to the present disclosure are bioresorbable and biocompatible, capable of long imaging time in vivo, and with a hepatic-biliary clearance mechanism, a combination of properties not found in any other nanoparticulate imaging agent or drug delivery system. The bioresorbability is triggered by the inherent pH changes associated with cellular endocytosis. In a preferred embodiment, the nanoparticles are calcium phosphosilicate nanoparticles (CPSNPs), designed to encapsulate imaging agents and/or therapeutics. The CPSNPs, the pegylated (PEG)-ICG-CPSNPs shown in FIGS. 2-6, are ideally suited to bioresorb in targeted tissue (based on targeting cancer tumors and cells up to this point), but circulate for long times in the blood stream if endocytosis does not take place, a property which is exploited according to the present disclosure for introduction into service members blood prior to entering combat conditions.

### Preparation of CPSNPs and Magnetite Nanoparticles (NPs)

The multimodal PEG-ICG-CPSNP contrast agent shown in FIG. 6(C), is prepared using synthetic and bioconjugation procedures previously described in U.S. Pat. No. 8,071,132, which is incorporated herein in its entirety. According to an embodiment of the disclosure, the core magnetite shown in FIG. 6(B) is synthesized using a modified synthetic procedure. The critical approach in the synthetic procedures for nanoparticles according to the present disclosure is to avoid irreversible agglomeration during synthetic steps as well as the laundering process that produces nanoparticle formulations suitable for IV injection currently used for animal trials or the oral formulations to be developed. The current synthetic scheme for the ICG-CPSNP using synthesis of the nanoscale calcium phosphosilicate employs a reverse micelle system composed of cyclohexane, p-nonyl-phenoxy-glycolether (Igepal CO-520), and calcium and phosphate-silicate aqueous solutions as the water phase. The silicate is present to stabilize the amorphous calcium phosphate phase to avoid crystallization of the nanoparticle material to one of the calcium phosphate crystalline phase, for example hydroxyapatite, Brushite, octacalcium phosphate, or similar compositions. The calcium containing reverse micelle is designated the A phase, the phosphate-silicate containing micelle designated the B phase, and mixture is the C phase. At a water to amphiphile molar ratio equal to about 2, the reverse micelle has a hydrodynamic diameter of about 10 nm. The exchange time for the C phase dictates the ultimate size of the resulting calcium phosphosilicate nanoparticles spherical nanoparticles as small as 10 nm diameter at 2.5 minutes or as large as 200 nm after 30 minutes of micellular exchange. Without seeking to be limited by a particular theory, it is believed particle growth according to the present disclosure proceeds by an agglomeration secondary nucleation mechanism. The micellular exchange is quenched by the addition of an aqueous solution of sodium citrate. The citrate molecule irreversibly adsorbs to the calcium phosphosilicate nanoparticles, permitting well-dispersed nanoparticle suspensions during subsequent laundering and provides carboxylate surface groups for ultimate bioconjugation.

In a further embodiment of the disclosure, the iron oxide nanoparticles shown in FIGS. 2-5 are synthesized using a modified Sugimoto-Matijevic procedure, with synthetic temperature at a more easily controlled 100° C. rather than 90° C. and with sodium citrate introduced with the reactants at temperature to control particle size and ensure well-dispersed magnetite nanoparticle suspensions. The carboxylate groups on the adsorbed citrate molecule also permit additional surface functionalization with the PEG moieties described below.

Once the nanoparticles are synthesized and functionalized with citrate, a packed bed laundering process, detailed in U.S. Pat. No. 8,071,132, herein incorporated by reference in its entirety, is used to remove spectator species and other residue from the reverse micelle synthesis exploiting reversible agglomeration of the CPSNPs. A polycarbonate or stainless-steel tube is packed with 200-micron spherical silica particles that act as the packed bed. The reverse micellular system is dissolved with 60 volume percent ethanol yielding a homogeneous solution composed of approximately 60 volume percent 95/5 ethanol/water, 40 volume percent cyclohexane, 8 weight percent amphiphile, and dissolved ions including sodium and chloride from the calcium, phosphate, and silicate salts. This homogeneous solution has a low dielectric constant and does not promote ionization meaning that the citric molecules on the surface of the CPSNPs are neutral. As the mixture is pumped through the packed silica column, the relatively large van der Waals attraction among the nanoparticles and the silica bed surfaces produces multiple layers of nanoparticles on the silica media. Additional ethanol is passed through the packed bed of silica and adsorbent nanoparticles until neither amphiphile nor cyclohexane are detected via UV-visible absorbance measurements. The nanoparticles are eluted from the packed bed of silica particles by a 70 volume percent ethanol and 30 volume percent water solution. The 70/30 ethanol-water permits charge formation on both the nanoparticles and silica particle surfaces resulting a net repulsive, electrosteric energy that displaces and dispersed the nanoparticles in the eluting solvent mixture. The ethanol present also ensures that the CPSNPs remain in a sterile state. The packed bed laundering reduces cyclohexane to less than 15 ppm (limit of detection of GC-MS), the amphiphile concentration changes from 8 weight percent to less than 5×10−4M, while maintaining the CPSNPs in a well dispersed state in the 70/30 ethanol-water solvent mixture.

The as-synthesized, citrate-dispersed magnetite nanoparticles are first washed with dilute nitric acid (10⁻³ M) to remove any oxidized iron oxide particulates and collected by centrifugation. The magnetite nanoparticles are repeatedly washed, centrifuged and resuspended in dilute sodium citrate solution (10⁻⁴ M) buffered to pH 7.4 until a constant specific conductivity is obtained indicating all extraneous species are reduced to negligible concentrations. With current centrifuge capabilities, a liter of concentrated magnetite nanoparticles can be washed by the iterative laundering approach. The final laundered citrate-magnetite suspension is redispersed in 70/30 ethanol-water for subsequent bioconjugation.

### Preparation of Oral Formulations

In one embodiment of the disclosure, oral formulations with enteric coatings to permit survival of the CPSNPs in stomach acidity, allowing NIR-CPSNPs to be incorporated in the diet of military personnel. Thus, the NIR-CPSNPs can be deployed either through intravenous injection on the battlefield or, given the relatively long circulation times, via food additives or tablets available to military personnel in meals, ready to eat (MRE).

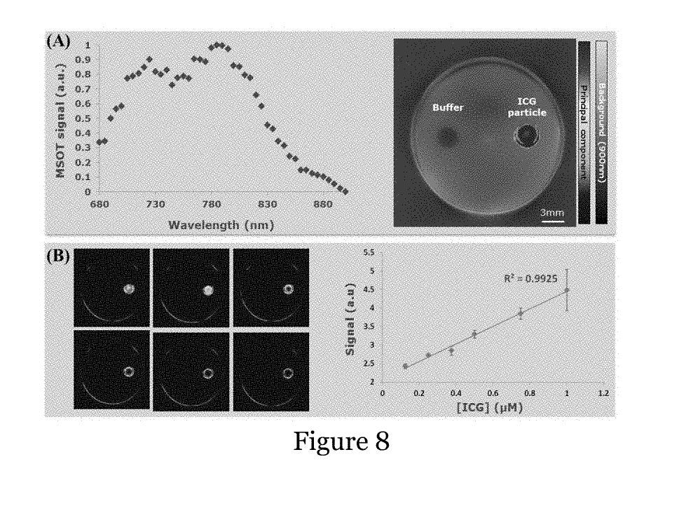

The basic matrix of the CPSNPs, calcium phosphate, is similar to that used in the commercial antacid, TUMS. According to the present disclosure, the criteria for nanoparticle drug delivery, criteria largely shared by nanoparticulates imaging contrast agents, include: 1. Improved contrast; 2. High resolution; 3. Inherently non-toxic materials and degradation products; 4. Small size (15 to 200 nm); 5. Encapsulation of active agent in a protective, impermeable matrix of calcium phosphosilicate; 6. Colloidally stable in physiological conditions; 7. Clearance mechanism; 8. Long clearance times; and, 9. Biologically or extrinsically controlled release of therapeutic agents. The NIR-CPSNPs according to the present disclosure meet all of these criteria. However, the photoacoustic CPSNPs of the present disclosure provide additional benefits for imaging systems, compared to the existing NIR-CPSNPs (FIGS. 6 and 8). The need for hierarchical imaging from the battlefield to the combat field facility to the large medical center to improve survival for wounded service members, demands the development of a multimodal imaging, nanoparticulate agent.

In a preferred embodiment of the disclosure, novel oral formulations are prepared for both the CPSNPs and the ensemble nanoparticles. The CPSNPs have been introduced in vivo in the murine animal models for cancer studies via systemic tail vein injection. In one aspect of the oral formulation, the ensemble contrast agent can be deployed in the diet of military personnel via MREs because of the biocompatibility of the formulations as well as the long circulation times. As a result, there will not be a need to introduce the ensemble nanoparticles via IV on the battlefield with the wait required for the ensemble nanoparticle contrast agent to distribute throughout the circulatory system of the wounded personnel. Thus, battlefield medical personnel can begin evaluation for internal trauma to almost immediately after an incident has taken place.

The production of oral formulations is well established and similar to the processing required for ceramic powders. Commercial polyacrylates are used as an enteric coating on the ensemble nanoparticles to inhibit dissolution during transport through the stomach of the animal models. Polyacrylic acid, the acidic form of polyacrylate, is insoluble in water at less than pH 4. Thus, the polyacrylate coatings permit the ensemble nanoparticles, particularly the CPSNPs, to remain intact, but as soon as the nanoparticles move into the small intestine where the pH is higher, the polyacrylate will dissolve and leave the PEG coated particles. The enteric acrylate can be introduced into the ensemble particle suspensions with the methylcellulose excipient. The suspension can be dried using high purity nitrogen in a sterile laminar flow hood followed by granulation to 1 mm in a grid gyratory granulator. Samples of the granules can then be dissolved and assayed for ICG, Ca, P, Si, and Fe concentrations to confirm the concentration of ensemble NPs on a dry weight basis.

### Preparation of IV Formulations

The standard procedure to prepare IV formulation is employed in this disclosure. The 70/30 ethanol-water suspension resulting from synthesis and bioconjugation of the functionalized nanoparticles is dried by flowing high purity nitrogen over the suspension in a sterile, laminar flow hood. The resulting nanoparticle aqueous suspension is diluted to a standard concentration equal to 1 μM ICG in sterile phosphate buffered saline (PBS, 10 mM sodium phosphate buffered to pH 7.4, 0.14 M NaCl, 0.01 M KCl). The CPSNPs are stable in PBS at 37° C. for extended times.

### Preparation of Ensemble, Multimodal Imaging Nanoparticles

The reaction used to bind the CPSNPs to a core magnetite NP as shown in FIG. 6(C) permits a large design space for the particle diameters as well as length of the molecular tethers used to bind the nanoparticles. The citrate-functionalized CPSNPs and citrate-magnetite NPs are functionalized with PEG via a carbodiimide linker molecule, ethyl-N-(3-dimethylaminopropyl)-N′hydrochloride carbodiimide (EDC). Reaction with the EDC produces a reactive, surface aldehyde group amenable to condensation reaction with either carboxylate or amine terminal groups on homogenous, linear PEG molecules. The formation of either ester or amide bonds, respectively, covalently links one end of the PEG molecule to the nanoparticle surface. The terminal group on the other end of the PEG exposed to the solution can either result in a well dispersed nanoparticle suspension with long circulation times in vivo (e.g., with a methoxy terminal group), or provide additional opportunities for bioconjugation. Two terminal groups that provide additional opportunities for bioconjugation are maleimide and sulfhydryl groups. The PEG-maleimide approach reacts stoichiometrically with terminal sulfhydryl on deca-gastrin via addition of the sulfide group and proton to the unsaturated bond in the maleimide ring. The deca-gastrin functionalized CPSNPs were shown to specifically target human pancreatic cancer.

In an embodiment of the disclosure, sulfhydryl terminated PEG with either carboxylate or amine on the opposite terminus is used to functionalize one of the citrate nanoparticles (CPSNPs or magnetite NPs) while maleimide terminated with either carboxylate or amine on the other terminus functionalizes the complementary nanoparticle material. Once the composite ensemble structure is prepared, any remaining, unreacted maleimide or sulfhydryl groups on the surfaces of the ensemble nanoparticles is reacted with short (less than 1 kD) sulfhydryl and maleimide terminal PEG with methoxy functionalization on the opposite terminus. According to the present disclosure, methoxy-terminated PEG on the outer surface of CPSNPs has shown that long circulation times (up to 96 hours) are achieved in vivo.

The relative concentration of the magnetite to the CPSNPs can be evaluated by IZON particle number counting before and after dissolution of the CPSNPs with lower pH 5 aqueous digestion for the CPSNPs. Thermogravimetric analysis up to 600° C. and lost on ignition at 105° C. is used to estimate the relative mass of magnetite and CPSNPs in each formulation before and after pyrolysis of organic material. Chemical analysis via inductively coupled argon plasma—mass spectroscopy may be employed to determine the relative concentration of magnetite and CPSNP in each ensemble formulation. Particle size distributions via the IZON electrical sensing zone technique, dynamic light scattering via the Brookhaven ZetaPALS system, and image analysis on the nanoparticles obtained via FE-SEM and TEM photomicrographs can be used to evaluate the architecture of the ensemble particles before and after dissolution of the CPSNPs attached to the surface of the magnetite NPs. Optical absorbance as a function of wavelength as well as fluorescence spectra can be obtained for selected samples, for example on a Gemini 96 well plate system and a PTI fluorimeter. Absorbance and fluorescence after dissolution of the CPSNPs can be used to verify that the ICG concentration and that the ICG was encapsulated in the CPSNPs.

### Photoacoustic Imaging Systems

The strong scattering of photons in soft tissue limits the depth of pure NIR imaging to a few centimeters. This prevents the use of optical methods to image deep into biological tissue to identify things such as malignant tissue or internal bleeding in real time. Acoustic waves, on the other hand, are only weakly scattered in soft tissue which makes them ideal for improving depth of imaging and are well-known in the medical community as ultrasound imaging. Pure ultrasound, however, suffers from poor contrast due to its reliance on the mechanical properties of biological tissues. In contrast, the use of photoacoustic methods according to the disclosure, as shown in FIG. 2, combines the high contrast of light absorption with improved depth imaging using ultrasound. The combination of NIR-fluorescence imaging for shallow depths and photoacoustic-generated ultrasound images for increasing depths results in a dual-imaging modality on the battlefield to improve real-time diagnosis of internal injuries.

The photoacoustic effect refers to the generation of ultrasonic waves due to the absorption of incident radiation. The ideal wavelengths in biological tissue are the near-infrared due to the increased depth of penetration. The penetration of light in biological tissue is dictated by the scattering and absorption characteristics of the tissue. Scattering in biological tissue is quite strong with the result being that even focused light beams (laser) quickly become diffuse within a short distance (≈mm) of the surface. This limits high-resolution imaging to these small depths. Beyond this depth, light propagation can be modeled by the diffusion law. Light absorption in biological tissue is dictated by the constituents, as shown in FIG. 3(A). Oxygenated and deoxygenated hemoglobin, as well as water, are strong absorbers, but their absorption characteristics are different as a function of wavelength, allowing their differentiation with optical imaging. The optimum wavelength for minimum absorption of radiation is about 800 nm. Other absorbing molecules such as ICG can be artificially introduced into the in vivo system to change absorption characteristics. According to an embodiment of the disclosure, engineered multimodal-imaging nanoparticles can be produced with an optimum combination of fluorescent molecules to improve the NIR-imaging capabilities near the surface and absorbing molecules to improve the acoustic signals generated for deeper imaging.

The absorption of incident radiation by these molecules results in a sufficient rise in temperature to produce an ultrasonic wave through the thermoelastic effect. To efficiently create photoacoustic signals, it is necessary to utilize very short pulses of light, shorter than the thermal and stress confinement times. The thermal confinement time is the time scale for dissipation of heat as a result of thermal conduction and can be approximated as τₜₕ≈Lₚ²/4D_{T}, where Lₚ is the characteristic linear dimension of the tissue structure of interest and D_{T} is the thermal diffusivity of the sample. The condition that the temporal length of the light pulse, Tₚ, be much less than the thermal confinement time effectively means that heat dissipation during the laser pulse is negligible. Similarly, the stress confinement time can be approximated as τₛ=Lₚ/c, where c is the speed of sound in the medium. If τₚ<τₛ, then thermoelastic stress can build up rapidly. If these conditions are met, then thermal expansion can cause a pressure rise, p₀, estimated by, p₀=(βc²/Cₚ)μ₀F=ΓA, where β is the isobaric volume expansion coefficient, Cₚ is specific heat, μa is the absorption coefficient, F is the local light fluence, A is the local energy deposition density and Γ is the Grüneisen coefficient, Γ=βc²/Cₚ. The increased pressure results in the initiation of an ultrasonic wave that can traverse the soft tissue.

In an aspect of the disclosure, acoustic waves in the MHz range have low scattering and deep penetration which is ideal for improved imaging at depths greater than possible with optical capabilities alone. Arrays of acoustic transducers convert the acoustic pulses to electrical signals which are then used to recreate the tissue structure images. The current state-of-the-art in photoacoustic imaging utilizes nanosecond-scale pulses from tunable (through the NIR range) laser sources with pulse energies of tens to hundreds of milliJoules. These systems typically consist of a high-energy Nd:YAG pump laser system operating at either the second (532 nm) or third (355 nm) third harmonics with repetition rates in the tens of Hz pumping an optical parametric oscillator (OPO) which can then produce tunable laser light.

According to an embodiment of the disclosure, to produce tunable light in the NIR, the second harmonic of the pump laser is used. In a further embodiment, the laser source is one which that produces much faster repetition rates and is a supercontinuum source which consists of a laser pulse passing through a photonic crystal fiber in which the nonlinear effects result in a highly broadened spectral bandwidth.

In a preferred embodiment of the disclosure, the system is a man-portable system with a hand-held accessory for real-time imaging of the body. This man-portable system, combined with dual-imaging modality nanoparticles would result in the capability to quickly identify regions and severity of internal bleeding for triage purposes. The disclosure demonstrates that size reduction in high-sensitivity NIR imaging units is possible, as is reduction in laser source size.

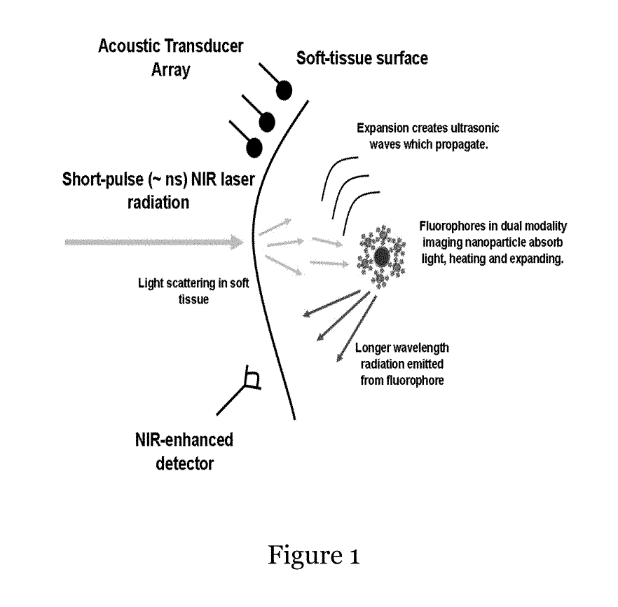

An embodiment of the disclosure comprises a rapid diagnostic approach based on a combination of NIR and photoacoustic imaging (FIG. 1) that, combined with the multimodal nanoparticle contrast agent according to the disclosure (FIG. 6), provide the following advantages: (1) Rapid, deep tissue detection of blood pooling and internal trauma; (2) Portability with the laser excitation source, for example housed within a rucksack worn by modern battlefield healthcare personnel; (3) Compatibility of the handheld device and nanoparticle contrast agent with treatment such as the battlefield foam and, in particular, designed to work in concert with internal hemorrhaging treatments; and, (4) Enhanced contrast for other imaging modalities as a function of distance from the battlefield and increasing sophistication of imaging and treatment for wounded service personnel.

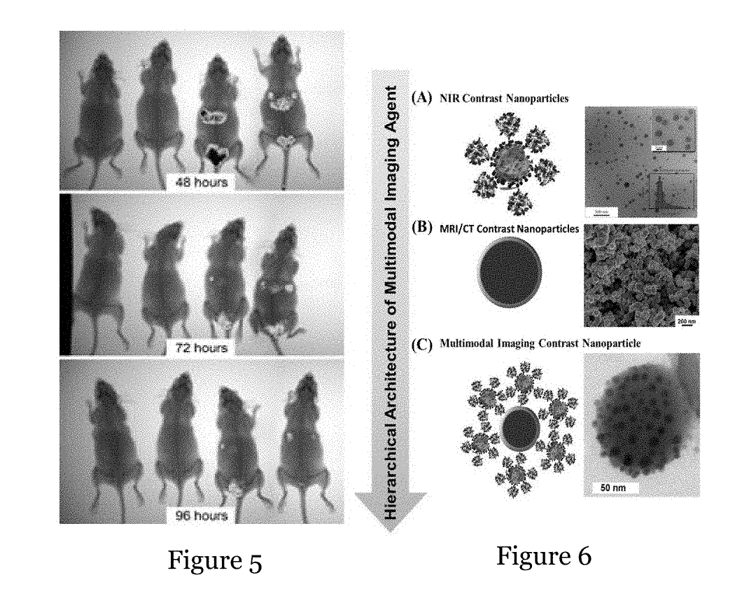

Alternatives to the NIR/photoacoustic approach are NIR tomography or ultrasound imaging. NIR tomography that can be used to a depth of several centimeters in real time will be deployed in concert with photoacoustic tomography for deeper soft tissue detection of internal bleeding based on measurements in human tissue. However, the tissue penetration and escape depth of the fluorescence photons are limited in real time (5 seconds or less of the excitation at 785 nm) to about 3 cm in porcine muscle tissue. Deeper tissue imaging is possible, but requires longer exposure times than the real time needed for rapid scanning and diagnosis on the battlefield. In contrast and according to the present disclosure, the miniaturization available with current and emerging laser technology mean that an excitation source for photoacoustic ultrasound is currently available or can be readily adapted using engineering approaches rather than the development of additional basic science. Furthermore, the nanoparticle contrast agents for early detection and treatment of cancer (described in U.S. Pat. No. 8,071,132, which is incorporated herein in its entirety) can be readily adapted for NIR/photoacoustic imaging, CT Scan and MRI as shown in FIG. 6. The nanoparticle NIR contrast agent according to the present disclosure with polyethylene(glycol) on the surface persists in the murine models used in animal trials to date for up to 96 hours (FIG. 5). Thus, NIR-photoacoustic with the nanoparticle contrast agents according to the present disclosure meets the four criteria required for rapid imaging and treatment of major battlefield trauma, particularly internal hemorrhaging.

### NIR Photoacoustic Imaging and Nanoparticulate Formulation Contrast Capabilities

The ensemble nanoparticle system of the present disclosure has features that permit tunability with respect to potential enhancement in both the NIR and NIR-photoacoustic spectra. The concept for the ensemble NIR nanoparticles was catalyzed by the inherent higher brightness than the NIR-CPSNPs associated with NIR Quantum Dots™ previously discussed. The ensemble of multiple ICG-CPSNPs is brighter than currently achieved with the individual CPSNPs so long as Förster distances, usually at no more than 1 to 3 nm, leading to self-quenching are exceeded with tethers of suitable length. However, recognizing the engineering constraints employed by multimodal imaging systems—as presented in Tables 2 and 3—both the luminescence and photoacoustic emission as a function of basic features of the multimodal, ensemble nanoparticles may be experimentally and theoretically evaluated. The particle diameters may be varied via synthetic control according to the present disclosure, in the case of CPSNPs, particle diameters from 10 to 200 nm can be obtained as a function of micellular equilibration time. For the magnetite NPs, particle diameter can be varied from 20 nm to 200 nm as a function of the iron and citrate concentrations used in the synthetic procedure. The PEG tether length will be varied from 1 to 5 nm based on the molecular weight of the PEG tether used to functionalize the nanoparticles. In an alternative embodiment, soluble collagen may be employed as a tether. In a further embodiment, collagen with an alpha helix conformation, vis-à-vis the random coil conformation of the PEG, may be exploited to utilize vibrational properties for the photoacoustic effects. In another embodiment, the concentration of ICG in each CPSNP may be altered, particularly for the photoacoustic spectra. In a preferred embodiment, between 6 to 8 molecules per nanoparticle in the standard, non-toxic formulation is obtained, but this concentration can be increased by increasing the micelle exchange time and/or modifying the adsorption of the ICG for the agglomerative growth by employing a greater ratio of Ca:P to increase local charge to bind the sulfonate groups on the ICG more strongly.

According to the present disclosure, the photoacoustic properties of pegylated, NIR-CPSNPs are is summarized in FIG. 8. The HSOT Signal vs. wavelength for the and the strong PA signal in FIG. 8(A) for the encapsulated PEG-ICG-CPSNP contrast agent are consistent with the enhanced NIR fluorescence spectra. The linear dose response for the PEG-ICG-CPSNPs in FIG. 8(B) has a lower limit of quantification 100 nM (77.5 nanoGrams/mL) consistent with the enhanced fluorescence emission reported for the indocyanine green encapsulated in the calcium phosphosilicate. (FIGS. 2-5).

The CPSNPs of the present disclosure combined with nanoscale magnetite, as shown in FIG. 6, create a nanoparticulate ensemble for enhanced contrast and resolution for NIR-photoacoustic, CT scan, and MRI imaging within one nanoparticulate. The ensemble nanoparticulates permit wide latitude in design and imaging optimization. In addition to the size and shape of the component particles, the tether length and the nature of the mechanical and vibrational properties of the tether molecular moieties (e.g., a linear random coil such as PEG molecules vis-à-vis a molecule with an alpha-helix conformation obtained with collagen tether molecules)—as well as the separation distance between magnetite core particles and NIR-CPSNPs as determined by different molecular weight tethers—can be used to ‘tune’ the photoacoustic properties of the individual ensemble nanoparticulate contrast agents. The properties associated with the individual nanoparticles, summarized in FIGS. 2-5 and 7, are exploited to enhance the multimodal imaging with the ensemble nanoparticulates of the present disclosure, shown in FIG. 6(C).

### Hybrid Multimode Portable Imaging System (HMPI)

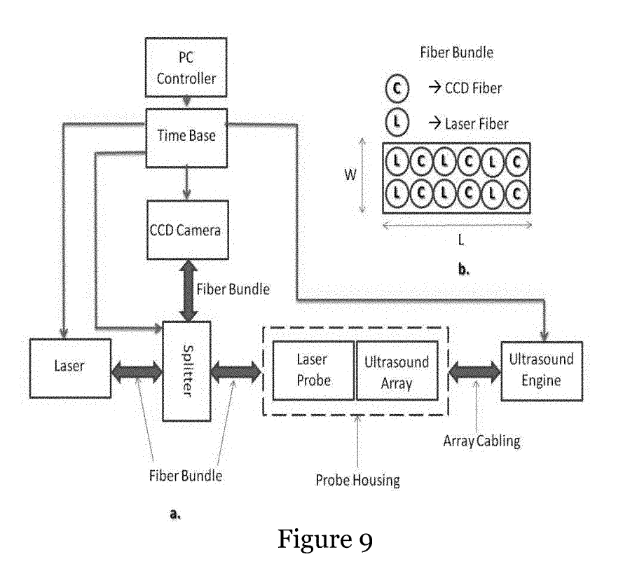

Another embodiment of the present disclosure comprises a hybrid imaging system for the purpose of detecting, enhancing and tracking the progression of custom designed nanoparticles in their role as agents to identify internal bleeding in combat casualty situations. A block diagram of the concept is depicted in FIG. 9.

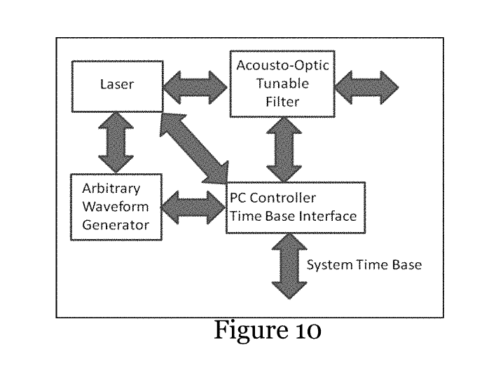

The hybrid system of the present disclosure, depicted in FIG. 9, combines a NIR imaging system utilizing a supercontinuum/tunable laser device combined with a photoacoustic tomography system. The hybrid approach of the present disclosure permits each specific imaging modality to make a significant contribution, while the combination compensates for the deficiencies related to each individual specific modality. This allows the user the capability, via the built in GUI, to control the specific complementary imaging sections. The laser consists of a wide bandwidth supercontinuum/tunable laser device. The laser system is illustrated in block diagram form in FIG. 10. The laser subsystem will receive its timing commands from the global controller illustrated in FIG. 9.

The subsystem comprises a time base interface which controls the selection of the tuning parameters for the acousto-optic filter that covers the bandwidth of the supercontinuum laser/OPO of the tunable laser, the arbitrary waveform generator that will be utilized in coded excitation experiments, and the laser timing and safety monitoring section. A fiber bundle is utilized to distribute the laser beam profile. The fiber bundle, shown in FIG. 9(b) is designed to perform two specific tasks: the first is to route the laser pulse to the probe handle in the photoacoustic mode (PA), and the second is to transmit NIR radiation and route the fluorescence NIR energy to the CCD camera in the NIR mode. This exemplary embodiment of the fiber bundle illustrates interleaved laser fibers, denoted by L, and CCD fibers denoted by C. The fiber bundle dimensions L and W are matched to the aperture of the ultrasound probe in the PA mode to uniformly illuminate the focal point of the ultrasound beam. The ultrasound engine is an Ultrasonix SonixTouch system which consists of a 128-channel research system with a parallel channel data acquisition system. The parallel channel data acquisition system is utilized to collect the raw RF reflected data.

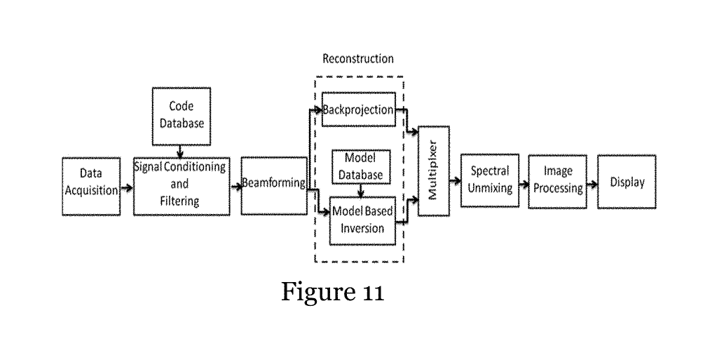

The signal processing chain is shown in FIG. 11. The extracted RF lines are processed by a signal conditioning and filtering stage, which has the ability to bandpass filter the signal channels to increase the signal to noise ratio as well as to be synchronized with the coded laser transmitted pulses (i.e., coded excitation processing consisting of minimal linear recursive sequence generation, Legendre polynomials) for correlation and pulse compression to be applied. The beam-forming stage provides the capability of performing advanced processing techniques such as fixed focusing, dynamic focusing, and dynamic apodization. Having the capability to vary the curvature of the wave front electronically will be an important tool that will be useful when studying the effects of new processing strategies related to combining ultrasonic modulation with optical phase conjugation such as TRUE (Time Reversed Ultrasonically Encoded optical focusing) and TROVE (Time Reversal Of Variance-Encoded Light).

The reconstruction stage permits two different approaches: filtered back-projection and model based inversion techniques. Back-projection algorithms, based on closed form inversion equations, can be implemented either in the spatio-temporal domain or in the Fourier domain. Since back-projection algorithms are derived based on ideal acoustic wave propagation conditions, generalization to varying optoacoustic illumination conditions is difficult. There has been a recent advance in model based inversion techniques that can be generalized to a set of matrix vector equations over a grid of spatial and temporal coordinates. Optimization algorithms that minimize the mean square error between the collected signal set and the predicted model representation set are used to converge to a solution. This vector matrix equation system is solved by Moore-Penrose pseudo inverse techniques. Both reconstruction methods will be implemented and tested. There will be a more focused effort on the model based approach utilizing the IMMI (i.e., interpolated—matrix model inversion) algorithm. An inference structure will be constructed that will add adaptability to the algorithm and potentially reconfigure the parameter sets associated with the physical attributes of the model.

The spectral unmixing algorithm is used to address the problem of mixed pixels in multi-spectral imagery. In a multi-spectral image, the measurement of a single pixel is usually a contribution from several materials called end members. The unmixing process comprises decomposition of mixed pixel spectra into end member signatures and their fractional abundances. The specific cases of interest are: Linear mixing, where the mixing scale is macroscopic and there is negligible interaction among distinct end members; and nonlinear mixing, where the mixing scale is microscopic, and the incident radiation scattered through multiple scattering events involves several end members. Unfortunately, most spectral mixtures observed in multispectral imagery have nonlinear mixing characteristics. Since most traditional unmixing techniques are based upon the linear mixing model, they perform poorly in finding the correct end members and their abundances in the case of nonlinear spectral mixing. Thus, in one aspect, the present disclosure utilizes unsupervised end member extraction techniques that include both ICA (Independent Component Analysis), and unsupervised blind signal deconvolution. In addition to the aforementioned methods, new nonlinear algorithms based on differential geometry allow for the calculation of geodesics in the nonlinear mixing model.

The image processing section of the present disclosure is used to implement advanced computer vision algorithms to the unmixed data sets. Specifically, the processing chain includes multidimensional edge preserving smoothing, segmentation, and non-linear feature extraction techniques. Edge preserving smoothing filters noise while preserving the edge detail in the image. Such techniques as bilateral filtering and diffusion based approaches will be studied. Current state of the art segmentation techniques using integrating features such as brightness, or texture over local image patches and then clustering those features based on fitting mixture models, mode-finding, or graph partitioning will be implemented. The ability to generate multispectral data sets will allow both shape, at a multi-scale level, and spectral signature to be used as a set of marker tools for detection, classification, and localization.

### Portable Probe Design

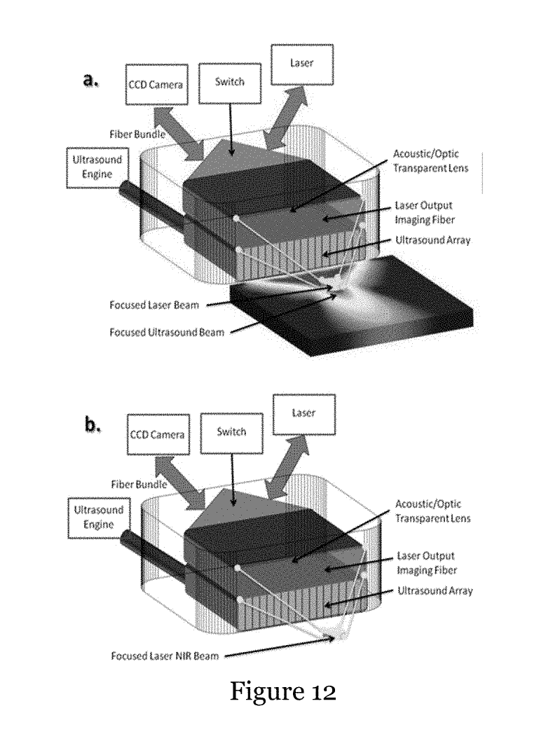

A preferred embodiment of the disclosure comprises a portable probe that comprises a multi-functional hybrid imaging system. This approach is illustrated in FIG. 12. FIG. 12(a) depicts the photoacoustic tomography (PAT) system with FIG. 10(b) the NIR system from the same probe. The actual functionality of the system has been described in the above sections and is illustrated in FIG. 9. A tradeoff table for the hybrid probe is shown in Table 1 while the cost rationale is shown in Table 2.

TABLE 1
The engineering compromises for hybrid system designs.
Imaging Technique/Benefit
DOT—Diffuse Optical Tomography

| | |
|---|---|
| 1. | Local fluence/Flux Calculation and Estimation |

PAT—Photoacoustic Tomography

| | |
|---|---|
| 1. | Optical Absorption Estimation |
| 2. | Ballistic or Quasi-Ballistic Imaging Depths |
| 3. | Micrometer Spatial Resolution |
| 4. | Works with Nanoparticles |
| 5. | Spectroscopic PAT: Oxygenated Hemoglobin and De-Oxygenated |
| | Hemoglobin estimation via Least Squares solution. Hemoglobin |
| | Oxygen Saturation estimation through back substitution of least |
| | squares results |

OCT—Optical Coherence Tomography

| | |
|---|---|
| 1. | Microstructure detection via Contrast Differential |
| 2. | Ballistic or Quasi-Ballistic Imaging Depths |
| 3. | Micrometer Spatial Resolution |
| 4. | Works with Nanoparticles |

NIR—Near Infrared

| | |
|---|---|
| 1. | High Photon Penetration |
| 2. | Reduced Light Scattering |
| 3. | Minimal Autofluorescence from Living Tissue |

TABLE 2
Overview of imaging systems for small animals.

| | | | | | | Training/ | |
|---|---|---|---|---|---|---|---|
| | | | | | | expertise | |
| Modality | Resolution | | Depth | Optimal use | Signal | required | Cost ⁺ |
| MRI | 10-100 | μm | No | Anatomical assessment, | RF * waves | Yes | $$$ |
| | | | limit | investigation of physiological, | (Nonionizing | (Certified | |
| | | | | metabolic, molecular and genetic | radiation) | radiologists) | |
| | | | | events. | | | |
| PET | 0.8-1.4 | mm | No | Investigation of physiological, | γ-rays | Yes | $$$ |
| | | | limit | metabolic, molecular and genetic | (Ionizing | (Certified | |
| | | | | events. | radiation) | radiologists) | |
| SPECT | 0.8-1.4 | mm | No | Investigation of physiological, | γ-rays | Yes | $$ |
| | | | limit | metabolic, molecular and genetic | (Ionizing | (Certified | |
| | | | | events. | radiation) | radiologists) | |
| CT | 50 | μm | No | Anatomical assessment. | X-rays | Yes | $$ |
| | | | limit | | (Ionizing | (Certified | |
| | | | | | radiation) | radiologists) | |
| Ultrasound | 50 | μm | mm | Anatomical assessment, | Sound waves | Yes | $$ |
| | | | | investigation of physiological, | (Nonionizing | (Certified | |
| | | | | metabolic, molecular and genetic | radiation) | sonographers) | |
| | | | | events. | | | |
| Fluorescence | 0.3 | μm | <1 cm | Metabolic, molecular and genetic | Light waves | No | $ |
| optical | | | | events. | (Nonionizing | | |
| imaging | | | | | radiation) | | |

MRI, Magnetic resonance imaging;
PET, Position emission tomography;
SPECT, Single photon emission computed tomography;
CT, Computed tomography;
* RF, radiofrequency,
⁺ Cost of system: $<100,000; $$100-300,000; $$$1-3 millions.

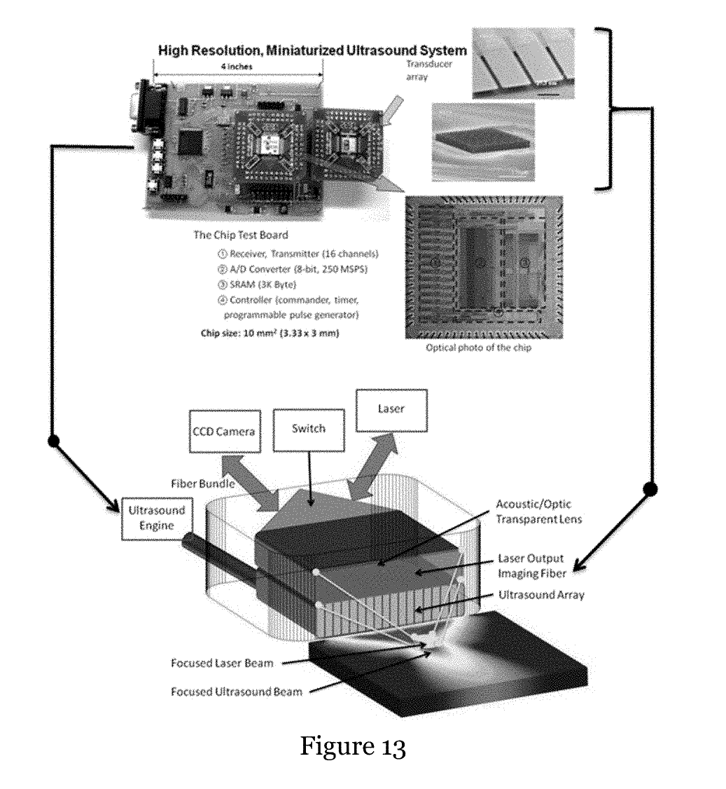

FIG. 13 illustrates the integrated product of a preferred embodiment of the disclosure. High-frequency ultrasound array transducers using piezoelectric thin films on larger structures are used for high-resolution imaging systems. The increase in resolution is achieved by a simultaneous increase in operating frequency and close coupling of the electronic circuitry. Two different processing methods were explored to fabricate array transducers. In one implementation, a xylophone bar transducer was prototyped, using a thin film as the active piezoelectric layer. In the other, the piezoelectric transducer was prepared by mist deposition of PZT films over electroplated Ni posts. Because the PZT films are excited through the film thickness, the drive voltages of these transducers are low, and close coupling of the electronic circuitry is possible. A complementary metal-oxide-semiconductor (CMOS) transceiver chip for a 16-element array was fabricated in 0.35 um process technology. The ultrasound front-end chip contains beam-forming electronics, receiver circuitry, and analog-to-digital converters with 3-Kbyte on-chip buffer memory.

Supercontinuum technology provides increased pulse repetition rate which is important to increase the imaging speed of the system, a critical component for real-time diagnosis. It also has increased flexibility in wavelength choices due to the high spectral bandwidth of the output. Multiple different types of fluorophores and/or absorbers with many different ideal wavelengths could be excited simultaneously, producing signals from multiple depths in the soft tissue and multiple species. However, present supercontinuum technology has considerably lower energy per pulse than the tunable OPO system which will result in reduced signal in both the NIR and photoacoustic regimes.

In a preferred embodiment of the disclosure, the system utilizes fiber-optic imaging bundle to improve fiber-optic delivery of both laser input light and NIR fluorescent light. The fiber-optic bundle consists of many individual fibers arranged in a geometric pattern which establishes the imaging area of the NIR detection system and the illumination area of the laser. This fiber bundle is integrated with an acoustic transducer into a single hand-held accessory that provides flexibility to image large areas quickly, such as the torso of a human being. This handheld accessory is designed for ease of use and to be integrated with existing medical computed tomography (CT) systems.

### Methods of Use

The present disclosure also relates to methods for detecting trauma. This embodiment of the methods can include administering to an individual or animal a nanoparticle bioimaging contrast agent for multimodal biological imaging according to the present disclosure. The administering can be provided in a number of ways depending on the specific formulation. IN an embodiment, the method further includes imaging the distribution of said nanoparticle bioimaging contrast agent within said individual or animal using a handheld photoacoustic, portable imaging system according to the present disclosure.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

### Example 1

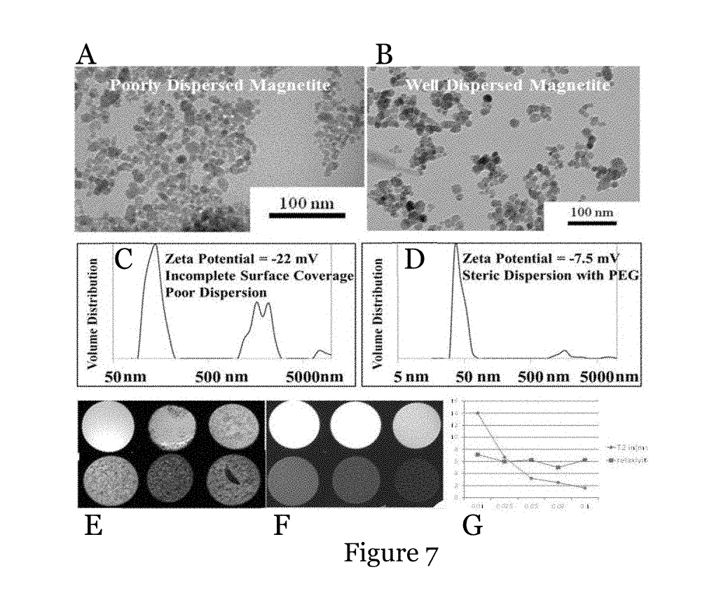

The magnetite particles of the present disclosure in FIG. 6 have been evaluated as a function of dispersion in MRI and are shown in FIG. 7. While the poorly dispersed magnetite nanoparticles show local hypointense signal areas within each image, the well dispersed magnetite nano-particles produce a uniform signal change over the whole image. It is of great importance to use well dispersed magnetite nanoparticles to be able to draw reliable conclusions about the local concentrations of these particles (e.g. in tissue). Using the poorly dispersed magnetite nanoparticles could over/under estimate the local concentrations. The phantom experiments showed a huge drop of the apparent T2 relaxation time from 96 ms in the Agar phantom without magnetite nanoparticles, to 14 ms with only 0.01 mg/ml magnetite nanoparticles. A further decrease to 1.6 ms was observed when 0.1 mg/ml was used. Acquired T2 maps showed very homogeneous distributions of T2 over the whole phantoms.

What is claimed is:

1. A system for rapid diagnosis of trauma comprising: a handheld photoacoustic, portable imaging system, comprising a tunable pulsed laser, a detector, and acoustic transducer arrays.

2. The system of claim 1 wherein said detector comprises CCD imaging technology.

3. The system of claim 1 wherein said pulsed laser comprises a high-energy Nd:YAG pump laser system or a supercontinuum source.

4. The system of claim 1 wherein said system can be transported by a single individual.

5. The system of claim 1, wherein said nanoparticle multimodal contrast agent comprises pegylated calcium phosphosilicate nanoparticles.

6. The system of claim 5, wherein said nanoparticle further comprises indocyanine green (ICG) and nanoscale magnetite.

7. A nanoparticle bioimaging contrast agent for multimodal biological imaging, comprising pegylated calcium phosphosilicate nanoparticles (PEG-CPSNPs).

8. The nanoparticle bioimaging contrast agent of claim 7, wherein said nanoparticle further comprises indocyanine green (ICG).

9. The nanoparticle bioimaging contrast agent of claim 7, wherein said nanoparticle further comprises nanoscale magnetite.

10. The nanoparticle bioimaging contrast agent of claim 7, wherein said agent is incorporated into a food additive or tablet.

11. The nanoparticle bioimaging contrast agent of claim 7, wherein said agent is incorporated into an IV formulation.

12. A method for rapidly detecting trauma in a person or animal, comprising
administering to said individual or animal a nanoparticle bioimaging contrast agent for multimodal biological imaging; and
imaging the distribution of said nanoparticle bioimaging contrast agent within said individual or animal using a handheld photoacoustic, portable imaging system.

13. The method of claim 12, wherein said nanoparticle bioimaging contrast agent comprises pegylated calcium phosphosilicate nanoparticles.

14. The method of claim 13, wherein said nanoparticle bioimaging contrast agent further comprises indocyanine green and nanoscale magnetite.

15. The method of claim 12, wherein said administration comprises providing said individual or animal with a food additive or tablet comprising said nanoparticle bioimaging contrast agent.

16. The method of claim 12, wherein said additive or tablet is administered daily.

17. The method of claim 12, wherein said administering is via an IV formulation.

18. The method of claim 12, wherein said handheld photoacoustic, portable imaging system comprises a tunable pulsed laser, a detector, and acoustic transducer arrays.

19. The method of claim 18, wherein said detector comprises CCD imaging technology.

20. The method of claim 18, wherein said pulsed laser comprises a high-energy Nd:YAG pump laser system or a supercontinuum source.
